(19) United States
(12) Patent Application Publication
(10) Pub. No.: US 2015/0315647 A1
(43) Pub. Date: Nov. 5, 2015

(54) MICROBIOME GENE EXPRESSION AS A BIOMARKER OF ARSENIC EXPOSURE

(71) Applicant: Barry P. ROSEN, Coral Gables, FL (US)

(72) Inventor: Barry P. ROSEN, Coral Gables, FL (US)

(73) Assignee: The Florida International University Board of Trustees, Miami, FL (US)

(21) Appl. No.: 14/704,323

(22) Filed: May 5, 2015

Related U.S. Application Data
(60) Provisional application No. 61/988,673, filed on May 5, 2014.

(51) Int. Cl. C12Q 1/68

(52) U.S. Cl. CPC C12Q 1/6883; C12Q 2600/158

(57) ABSTRACT

The present invention provides methods and kits to screen for arsenic exposure. The methods and kits utilize quantitative (qPCR) and/or other bioassays to analyze levels of arsenic-inducible genes and reporter genes as biomarkers for exposure.

### CROSS-REFERENCE TO RELATED APPLICATIONS

This application claims the priority benefit of U.S. Provisional Application Ser. No. 61/988,673, filed May 5, 2014, which is incorporated herein by reference in its entirety.

### GOVERNMENT SUPPORT

The subject invention was made with government support under a research project supported by the National Institutes of Health, Grant No. GM55425. The government has certain rights in this invention.

### BACKGROUND OF INVENTION

Arsenic is a pervasive human carcinogen. There is a considerable need for biomarkers of exposure in human populations exposed to levels of arsenic exceeding the EPA/WHO MCL of 10 ppb. Current “biomarkers” include measurement of levels of inorganic and methylated arsenic species in blood, urine and feces. Detection requires expensive instrumention, is slow, and only indirectly measures exposure.

Bacteria and fungi have genes involved in arsenic detoxification that are inducible by arsenic. More than a dozen genes have been identified that are induced by ppb levels of arsenic. Many of these genes are present in the bacteria and fungi that comprise the human gut, dermal and respiratory microbiomes.

### BRIEF SUMMARY

The present invention provides methods and kits for screening for arsenic exposure.

In one aspect, the present invention provides methods of detecting arsenic exposure in a subject, comprising: receiving a sample collected from the subject, the sample having at least one of a bacterium or fungus comprising an arsenic-inducible gene; extracting RNA from the sample; carrying out a quantitative PCR reaction on the RNA with primers specific for at least one of the bacterium or fungus arsenic-inducible gene; and detecting a PCR amplicon. The presence of the PCR amplicon indicates arsenic exposure.

In another aspect, the present invention provides methods of detecting arsenic exposure in a subject, comprising: receiving a sample collected from the subject, the sample having bacteria carrying at least one arsenic-inducible reporter gene; extracting RNA from the sample; carrying out a quantitative PCR reaction on the RNA with primers specific for the at least one arsenic-inducible reporter gene; and detecting a PCR amplicon. The presence of the PCR amplicon indicates arsenic exposure.

In another aspect, the present invention provides methods of detecting arsenic exposure in a subject, comprising: analyzing a sample collected from the subject, wherein the sample has at least one of a bacterium or fungus comprising an arsenic-inducible reporter gene; and detecting and/or quantitating the expression of the arsenic-inducible reporter gene. The presence of an expression signal over a certain control baseline signal indicates arsenic exposure.

In another aspect, the present invention provides kits for the detection of arsenic exposure in a cell, the kits comprising a primer set having a forward primer and a reverse primer, wherein the forward primer and the reverse primer are capable of generating a PCR amplicon from a region of one or more bacterial or fungal arsenic-inducible genes.

In some embodiments, the sample may be a fecal specimen for analyzing for oral exposure, a dermal bacteria specimen for analyzing for skin exposure, or a nasal swab specimen for analyzing for inhalation exposure. In other embodiments, the sample is any specimen obtained from a subject that has capacity to have bacteria and/or fungi growing thereon/therein. In some embodiments, the arsenic-inducible gene is a bacterial gene selected from As(III)-translocating efflux pump subunit (ArsA), As(III)-translocatingefflux pump subunit (ArsB), As(V) reductase (ArsC), As(III) chaperone (ArsD), MAs(III) oxidase (ArsH), MAs(III) demethylase (ArsI), As(III) methyltransferase (ArsM), As(III)-responsive transcription factor (ArsR), and combinations thereof. In other embodiments, the arsenic-inducible gene (or arsenic-inducible reporter gene) is a fungal gene selected from As(III)-responsive transcription factor (Acr1), As(V) reductase (Acr2), As(III) efflux permease (Acr3), and combinations thereof.

In some embodiments, the arsenic-inducible reporter gene comprises a promoter from a gene selected from As(III)-translocating efflux pump subunit (ArsA), As(III)-translocatingefflux pump subunit (ArsB), As(V) reductase (ArsC), As(III) chaperone (ArsD), MAs(III) oxidase (ArsH), MAs(III) demethylase (ArsI), As(III) methyltransferase (ArsM), As(III)-responsive transcription factor (ArsR), and combinations thereof In other embodiments, the arsenic-inducible reporter gene comprises a promoter from a gene selected from As(III)-responsive transcription factor (Acr1), As(V) reductase (Acr2), As(III) efflux permease (Acr3), and combinations thereof.

### BRIEF DESCRIPTION OF THE DRAWINGS

FIG. 1 shows relative fluorescence of arsenic-inducible green fluorescent protein from various arsenic-inducible reporter genes.

### DETAILED DISCLOSURE

Reference is made to particular features (including method steps) of the invention. The disclosure of the invention in this specification includes all possible combinations of such particular features. For example, where a particular feature is disclosed in the context of a particular aspect or embodiment of the invention, that feature can also be used, to the extent possible, in combination with and/or in the context of other particular aspects and embodiments of the invention, and in the invention generally.

The present invention provides methods and kits to screen for arsenic exposure. The methods and kits utilize quantitative (qPCR) and/or other bioassays to analyze levels of arsenic-inducible genes and reporter genes as biomarkers for exposure. A number of these genes code for enzymes that metabolize arsenicals, and their expression can serve as biomarkers of arsenic biotransformation into more toxic and carcinogenic species. Furthermore, biosensors may be utilized alone or in combination with qPCR to assay the gene expression. Such biomarkers of arsenic exposure can be used diagnostically for susceptibility to arsenic-related diseases including lung cancer, bladder cancer, skin cancer, and cardiovascular and neurological diseases.

In one aspect, the present invention provides methods of detecting arsenic exposure in a subject, comprising: receiving a sample collected from the subject, the sample consisting of at least one of a bacterium or fungus comprising an arsenic-inducible gene; extracting RNA from the sample; carrying out a quantitative PCR reaction with primers specific for at least one of the bacterium or fungus arsenic-inducible genes; and detecting a PCR amplicon. The presence of the PCR amplicon indicates arsenic exposure.

In another aspect, the present invention provides methods of detecting arsenic exposure in a subject, comprising: receiving a sample collected from the subject, the sample consisting of at least one of a bacterium or fungus comprising an arsenic-inducible gene or arsenic-inducible reporter gene; extracting nucleic acids from the sample; carrying out a quantitative reaction on the nucleic acids with one or more probes specific for at least one of the bacterium or fungus arsenic-inducible gene or reporter gene; detecting a signal; and quantitating the signal. The presence of a signal over a certain control baseline indicates arsenic exposure.

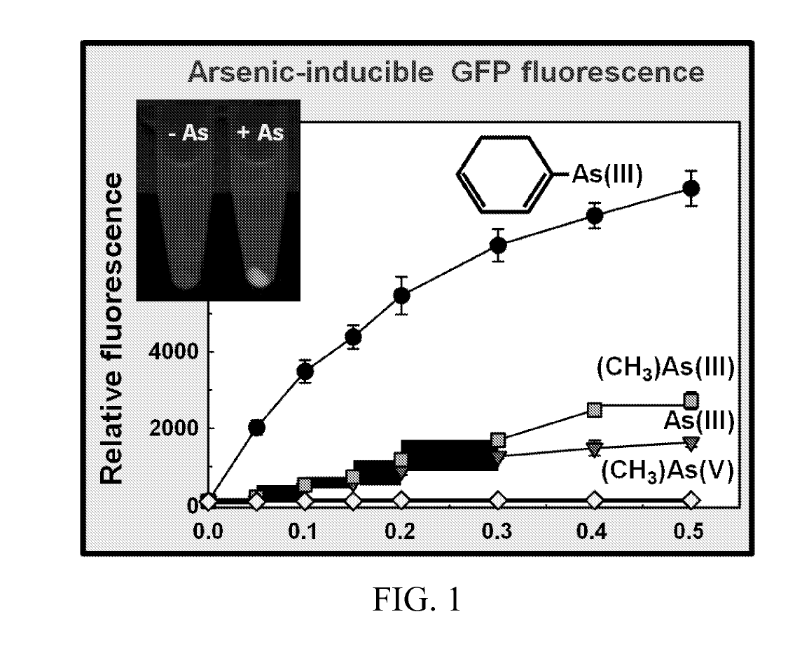

In another aspect, the present invention provides methods of detecting arsenic exposure in a subject, comprising: receiving a sample collected from the subject, the sample consisting of at least one of a bacterium or fungus comprising an arsenic-inducible reporter gene; and detecting and/or quantitating the expression of the arsenic-inducible reporter gene. The presence of an expression signal over a certain control baseline signal indicates arsenic exposure. FIG. 1 illustrates the ability of arsenic to stimulate expression of green fluorescent protein from arsenic-inducible GFP reporter genes.

The arsenic-inducible reporter gene(s) may be introduced into the microbiome of a subject via innocuous bacteria carrying the reporter gene(s). The sample is subsequently collected for arsenic exposure detection.

In some embodiments, the arsenic-inducible reporter gene comprises a promoter element from a gene selected from the group consisting of As(III)-translocating efflux pump subunit (ArsA), As(III)-translocatingefflux pump subunit (ArsB), As(V) reductase (ArsC), As(III) chaperone (ArsD), MAs(III) oxidase (ArsH), MAs(III) demethylase (ArsI), As(III) methyltransferase (ArsM), As(III)-responsive transcription factor (ArsR), and combinations thereof In other embodiments, the arsenic-inducible reporter gene comprises a promoter from a gene selected from the group consisting of As(III)-responsive transcription factor (Acr1), As(V) reductase (Acr2), As(III) efflux permease (Acr3), and combinations thereof The reporter gene may be any reporter known in the art, such as for example, but not limited to, green fluorescent protein, and is functionally linked to one or more promoter elements of the bacterial and/or fungal genes described herein.

In another aspect, the present invention provides methods of detecting arsenic exposure in a subject, comprising: receiving a sample collected from the subject, the sample consisting of bacteria carrying at least one arsenic-inducible reporter gene; extracting RNA from the sample; carrying out a quantitative PCR reaction on the RNA with primers specific for the at least one arsenic-inducible reporter gene; and detecting a PCR amplicon. The presence of the PCR amplicon indicates arsenic exposure.

In another aspect, the present invention provides kits for the detection of arsenic exposure in a cell, the kits comprising a primer set consisting of a forward primer and a reverse primer, wherein the forward primer and the reverse primer are capable of generating a PCR amplicon from a region of one or more bacterial or fungal arsenic-inducible genes.

In some embodiments, the sample may be a fecal specimen for analyzing for oral exposure, a dermal bacteria specimen for analyzing for skin exposure, or a nasal swab specimen for analyzing for inhalation exposure. In other embodiments, the sample is any specimen obtained from a subject that has capacity to have bacteria and/or fungi growing thereon/therein.

In some embodiments, the arsenic-inducible gene is a bacterial gene selected from the group consisting of As(III)-translocating efflux pump subunit (ArsA), As(III)-translocatingefflux pump subunit (ArsB), As(V) reductase (ArsC), As(III) chaperone (ArsD), MAs(III) oxidase (ArsH), MAs(III) demethylase (ArsI), As(III) methyltransferase (ArsM), As(III)-responsive transcription factor (ArsR), and combinations thereof. In other embodiments, the arsenic-inducible gene is a fungal gene selected from the group consisting of As(III)-responsive transcription factor (Acr1), As(V) reductase (Acr2), As(III) efflux permease (Acr3), and combinations thereof.

The term “subject,” as used herein, describes an organism, including mammals, for which the methods and kits according to the subject invention can be utilized. Mammalian species that can benefit from the disclosed methods and kits include, but are not limited to, apes, chimpanzees, orangutans, humans, monkeys; and other animals such as dogs, cats, horses, cattle, pigs, sheep, goats, chickens, mice, rats, guinea pigs, and hamsters.

It should be understood that the examples and embodiments described herein are for illustrative purposes only and that various modifications or changes in light thereof will be suggested to persons skilled in the art and are to be included within the spirit and purview of this application.

What is claimed is:

1. A method of detecting arsenic exposure in a subject, comprising:
receiving a sample collected from the subject, the sample having at least one of a bacterium or fungus comprising an arsenic-inducible gene;
extracting RNA from the sample;
carrying out a quantitative PCR reaction on the RNA with primers specific for at least one bacterium or fungus arsenic-inducible gene; and
detecting a PCR amplicon;
wherein the presence of the PCR amplicon indicates arsenic exposure.

2. The method of claim 1, wherein the sample is a fecal specimen for analyzing for oral exposure.

3. The method of claim 1, wherein the sample is a dermal bacteria specimen for analyzing for skin exposure.

4. The method of claim 1, wherein the sample is a nasal swab specimen for analyzing for inhalation exposure.

5. The method of claim 1, wherein the arsenic-inducible gene is a bacterial gene selected from the group consisting of As(III)-translocating efflux pump subunit (ArsA), As(III)-translocatingefflux pump subunit (ArsB), As(V) reductase (ArsC), As(III) chaperone (ArsD), MAs(III) oxidase (ArsH), MAs(III) demethylase (ArsI), As(III) methyltransferase (ArsM), As(III)-responsive transcription factor (ArsR), and combinations thereof.

6. The method of claim 1, wherein the arsenic-inducible gene is a fungal gene selected from the group consisting of As(III)-responsive transcription factor (Acr1), As(V) reductase (Acr2), As(III) efflux permease (Acr3), and combinations thereof.

7. A kit for the detection of arsenic exposure in a cell, the kit comprising a primer set comprising a forward primer and a reverse primer, wherein the forward primer and the reverse primer are capable of generating a PCR amplicon from a region of one or more bacterial or fungal arsenic-inducible genes.

8. The kit of claim 7, wherein the arsenic-inducible gene is a bacterial gene selected from the group consisting of As(III)-translocating efflux pump subunit (ArsA), As(III)-translocatingefflux pump subunit (ArsB), As(V) reductase (ArsC), As(III) chaperone (ArsD), MAs(III) oxidase (ArsH), MAs(III) demethylase (ArsI), As(III) methyltransferase (ArsM), As(III)-responsive transcription factor (ArsR), and combinations thereof.

9. The kit of claim 7, wherein the arsenic-inducible gene is a fungal gene selected from the group consisting of As(III)-responsive transcription factor (Acr1), As(V) reductase (Acr2), As(III) efflux permease (Acr3), and combinations thereof.

10. A method of detecting arsenic exposure in a subject, comprising:
analyzing a sample collected from the subject, wherein the sample has at least one bacterium or fungus comprising an arsenic-inducible reporter gene; and
detecting and/or quantitating the expression of the arsenic-inducible reporter gene, wherein the presence of expression over a certain control baseline indicates arsenic exposure.

11. The method of claim 10, wherein the arsenic-inducible reporter gene comprises a promoter element from a bacterial gene selected from the group consisting of As(III)-translocating efflux pump subunit (ArsA), As(III)-translocatingefflux pump subunit (ArsB), As(V) reductase (ArsC), As(III) chaperone (ArsD), MAs(III) oxidase (ArsH), MAs(III) demethylase (ArsI), As(III) methyltransferase (ArsM), As(III)-responsive transcription factor (ArsR), and combinations thereof.

12. The method of claim 10, wherein the arsenic-inducible reporter gene comprises a promoter element from a fungal gene selected from the group consisting of As(III)-responsive transcription factor (Acr1), As(V) reductase (Acr2), As(III) efflux permease (Acr3), and combinations thereof.
